(19) United States
(12) Patent Application Publication
(10) Pub. No.: US 2021/0398679 A1
(43) Pub. Date: Dec. 23, 2021

(54) SYSTEMS AND METHODS FOR USING MULTISCALE DATA FOR VARIABLE, PATHWAY, AND COMPOUND DETECTION

(71) Applicants: Northwestern University, Evanston, IL (US); Purdue Research Foundation, West Lafayette, IN (US)

(72) Inventors: Sumra BARI, Evanston, IL (US); Hans C. BREITER, Evanston, IL (US); Eric NAUMAN, West Lafayette, IN (US); Tom TALAVAGE, West Lafayette, IN (US); Nicole VIKE, Evanston, IL (US)

(73) Assignees: Northwestern University, Evanston, IL (US); Purdue Research Foundation, West Lafayette, IN (US)

(21) Appl. No.: 17/352,046

(22) Filed: Jun. 18, 2021

Related U.S. Application Data
(60) Provisional application No. 63/041,609, filed on Jun. 19, 2020.

(51) Int. Cl. G16H 50/20; G16B 40/20; G16B 50/10; G16B 50/30; G16H 10/40; G16H 50/50

(52) U.S. Cl. CPC G16H 50/20; G16B 40/20; G16H 50/50; G16B 50/30; G16H 10/40; G16B 50/10

(57) ABSTRACT

A method can apply permutation procedures to mediation and moderation tests of multiple hypotheses, while controlling the rate of false positives. The techniques presented here through a platform-independent tool can be applied to a variety of datasets in diverse and interdisciplinary fields, such as biology and medicine, where integration of multi-scale data is utilized to unmask disease diagnosis, prognosis, susceptibility/resilience, treatment optimization, and biopharmaceutical development for any brain-based, psychological, or medical illness. This platform allows for study of human illness where animal models are proving inadequate.

### CROSS-REFERENCE TO RELATED APPLICATIONS

This application claims priority to U.S. Provisional Application No. 63/041,609, filed Jun. 19, 2020, the entire contents of which are incorporated herein by reference.

### BACKGROUND

Multi-scale data sets can include data for various types of parameters, and may vary over time, such as by being from longitudinal studies. For example, multiscale data sets can include data from behavior of a subject and brain cells of the subject.

### SUMMARY

Multi-scale data can be used in a permutation-based mediation/moderation statistical framework for identifying variables, pathways, and compounds that can be used for illness diagnosis, prognosis, susceptibility/resilience prediction, treatment optimization, and biopharmaceutical development for any brain-based, psychological, or medical illness.

Multi-scale data is becoming increasingly important to discover new biomarkers for the diagnosis and prognosis of disease. Multi-scale datasets can include measurements of several variables either collected longitudinally or across different groups, such as to integrate variables collected from discrete spatio-temporal levels of organization, for instance between individual behavior and distributed groups of cells in the brain (e.g., from experimental psychology and brain imaging), or from groups of cells (via neuroimaging) and genotype. For example, multi-scale data can include various neuro-imaging measures, transcriptomics, metabolomics, behavioral measures at the individual level, or social variables that reflect group behavior. It can be difficult to analyze and integrate these datasets and make inferences that are replicable and generalizable to the larger population or disease/disorder subgroup due to factors such as small sample sizes (n<100).

Mediation and moderation methods are useful statistical methods that can be used to assess directional causation and variable dependencies across multi-scale data (e.g., three-way regression associations between metabolite measures, miRNA levels, and behavioral scores where one variable carries the relationship between the other two or where an interaction between two variables can be quantified in its effect on the third). These types of analyses can be done in any number of dimensions, including three-way relationships. For example, mediation can clarify the causal relationship between the independent variable (IV) and dependent variable (DV) with the inclusion of a third variable mediator (M). The mediation model proposes that instead of a direct causal relationship between IV and DV, the IV influences M which then influences the DV (e.g., the M “carries” the relationship between IV and DV). The moderation model proposes that the strength and direction of the relationship between independent variable (IV) and dependent variable (DV) is controlled by the moderator variable (M). Such methods can tie together multi-scale biological data, such as to identifying that variables which can be manipulated (e.g., molecular pathways) are related to brain pathology and behavioral dysfunction as symptoms or signs of illness. These types of integrative methods can form the basis for determining disease diagnosis and/or prognosis. Despite this potential, it can be difficult to correct for multiple comparisons across these scales given 1) limited and small sample sizes and 2) the fact that too stringent of corrections can result in many false negatives. Systems and methods in accordance with the present disclosure can enable novel permutation-based mediation and moderation methods to analyze and integrate multi-scale datasets while correcting for multiple comparisons.

Permutation-based methods can be useful to make distribution-free inferences and to control for the occurrence of false positives due to multiple hypothesis testing. Permutation tests can re-sample observations from the original data multiple times to build empirical estimates of the null distribution for the test statistic being studied. Smaller sample size studies using parametric tests can require assumptions about the underlying distribution of the data, which can make inferences difficult to replicate. Instead, permutation-based tests can be well-suited for studies with small sample sizes as they estimate the statistical significance directly from the data being analyzed rather than making assumptions about the underlying distribution. First, the test statistic is obtained from the original data set, then the data is randomly permuted multiple (Q) times and the test statistic is computed on each permutated data set. The statistical significance is computed by counting (K) the number of times the statistic value obtained in the original data set was more extreme than the statistic value obtained from the permuted data sets, and dividing that value by the number of random permutations (K/Q).

Systems and methods in accordance with the present disclosure can be applied to a longitudinal multi-scale dataset with measures scaling the transcriptome, the metabolome, resting-state brain networks, and virtual reality behavior. In an example, all measures were collected at two time points for 17 collegiate-level American football athletes. The developed permutation-based mediation and moderation methods assisted with the discovery of complex relationships between the aforementioned measures in this small cohort of football athletes as a function of repetitive mechanical accelerations to the head. For example, metabolic pathways focused on mitochondria can be identified.

The permutation-based mediation and moderation methods can 1) help to fill the gap in methodology required to integrate multi-scale datasets with several measures for small sample sized studies and 2) control for the occurrence of false positives due to multiple hypothesis testing. This platform can be useful for identifying molecular pathways at the core of human illness/disorder. This can be useful for fields where animal models have been inadequate for determining what is the core set of problems for human disease, such as psychiatry and some neurological illnesses (e.g., Parkinson's Disease).

At least one aspect relates to a method for determining which biological and psychological variables are fundamental for illness or injury diagnosis. The method can include receiving samples from a plurality of scales of organization in at least one of a body or a brain of one or more subjects; generating a quantitative output using the samples; determining that one or more compounds corresponding with the samples are increased or decreased relative to demographically matched normative controls responsive to the quantitative output; determining that an individual has a variable profile similar to a subject having a target illness responsive to at least one of the determination of increase or decrease or the quantitative output; integrating at least one first variable using a permutation-based mediation and moderation process to compute a first diagnostic likelihood; integrating an output of performing the permutation-based mediation and moderation process with at least one second variable to at least one of update the first diagnostic likelihood or compute a second diagnostic likelihood; and using a plurality of measures from multiple levels of spatio-temporal organization with machine learning to predict diagnosis.

At least one aspect relates to a method of prognosing the longitudinal course of an illness or injury. The method can include receiving samples from a plurality of scales of organization in at least one of a body or a brain of one or more subjects; determining that one or more compounds corresponding with the samples are increased or decreased relative to demographically matched normative controls; determining, responsive to the determination of increase or decrease, a longitudinal course of an individual when the person has a variable profile similar to that of a subject having an illness or injury for which a course of recovery is known; integrating at least one first variable using a permutation-based mediation and moderation process to compute a prognostic likelihood; integrating an output of performing the permutation-based mediation and moderation process with at least one second variable to compute a prognostic likelihood; and using a plurality of measures from multiple levels of spatio-temporal organization with machine learning to predict prognosis for the longitudinal course of an individual with the illness or injury.

At least one aspect relates to a method of assessing susceptibility for and resilience against an illness or injury. The method can include receiving samples from a plurality of scales of organization in at least one of a body or a brain of one or more subjects; generating a quantitative output using the samples; determining that one or more compounds corresponding with the samples are increased or decreased relative to demographically matched normative controls; determining, responsive to the determination of increase or decrease, that an individual has a variable profile in a range predicting susceptibility for and resilience against an illness or injury; integrating at least one first variable using permutation-based mediation and moderation process to assess susceptibility for and resilience against an illness or injury; integrating at least one second variable using an output of the permutation-based mediation and moderation process to assess susceptibility for and resilience against an illness or injury; and using a plurality of measures from multiple levels of spatio-temporal organization with machine learning for assessing susceptibility for and resilience against an illness or injury.

At least one aspect relates to a method of determining the optimal treatment for an illness or injury. The method can include receiving samples from a plurality of scales of organization in at least one of a body or a brain of one or more subjects; generating a quantitative output using the samples; determining that one or more compounds corresponding with the samples are increased or decreased relative to demographically matched normative controls; determining, responsive to the determination of increase or decrease, that an individual has an illness or injury profile consistent with individuals for which a particular treatment of an illness or injury has satisfied a treatment criteria; integrating at least one first variable using a permutation-based mediation and moderation process to assess optimal treatment for an illness or injury; integrating at least one second variable with an output of the permutation-based mediation and moderation process to determine the optimal treatment for an illness or injury; and using a plurality of measures from multiple levels of spatio-temporal organization with machine learning for determining the optimal treatment for a concussion or head injury.

At least one aspect relates to a method of determining the optimal point in a pathway or process for identifying if a biopharmaceutical compound may minimize the metabolomic, transcriptomic, or proteomic abnormalities or other variables quantifying the illness or injury. The method can include quantifying if metabolomic measures are altered in a therapeutic, prognostic, predictive manner for individuals with an illness or injury; determining if the biopharmaceutical compound alters metabolomic, proteomic, transcriptomic or other variables more than demographically matched normative controls; assessing if the biopharmaceutical compound affects a plurality of measures so an individual has a metabolomic, proteomic, transcriptomic or other variables profile consistent with individuals that have responded well to a particular treatment of that illness or injury; testing the biopharmaceutical compound against integrated variable indices for optimal treatment of an illness or injury; testing the biopharmaceutical compound against metabolomic, proteomic, transcriptomic or other variable data with hormone measures (e.g., progesterone) for optimal treatment of an illness or injury; testing the biopharmaceutical compound against metabolomic, proteomic, transcriptomic or other variable data with genotype data (e.g., a SNP at DARC or TPH2 or KIAA0319) for the optimal treatment of an illness or injury; and testing the biopharmaceutical compound against a plurality of measures from metabolomic, transcriptomic, proteomic, hormone, genetics data with machine learning for optimal treatment of an illness or injury.

At least one aspect relates to a system that includes one or more processors. The one or more processors can be configured to perform at least a portion of one or methods described herein.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIGS. 1A-1C depict example models and charts of methods for mediation methods for multiscale data processing; and

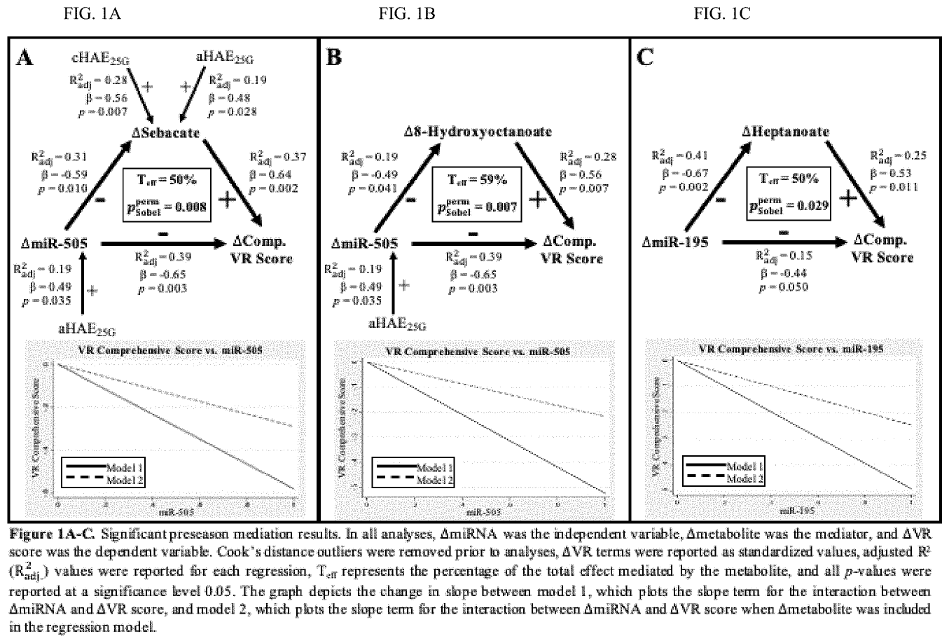

FIGS. 2A-2C depict example models and charts of methods for moderation methods for multiscale data processing.

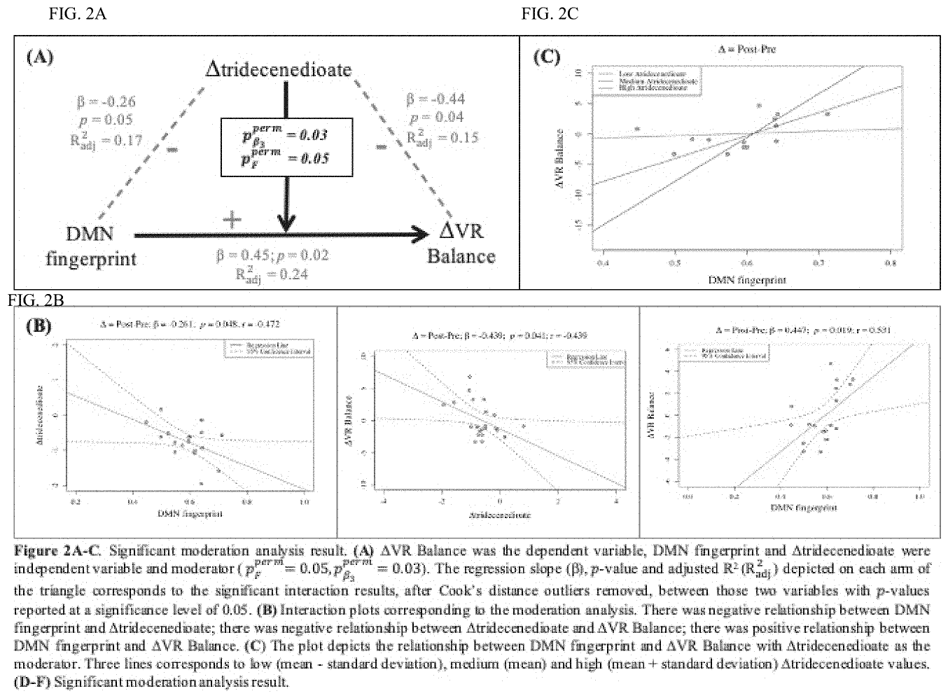

### DETAILED DESCRIPTION

Systems and methods in accordance with the present disclosure can implement 1) identification of two-way associations, 2) discovery of their overlap as three-way associations, and 3) mediation and moderation testing within a permutation-based framework. An example of a generalized framework is described here for three groups of measurements with different numbers of variables in each group acquired at two points for all participants. This can be extended to more groups and timepoints.

The measurement matrices Xₜ, Yₜ, Zₜ defined below schematize the variables used.

$${X}_{t} = [ \begin{matrix}{x}_{t , 1 , 1} & & {x}_{t , N , 1} \\ ⋮ & … & ⋮ \\ {x}_{t , 1 , S} & & {x}_{t , N , S}\end{matrix} ] , \\ {Y}_{t} = [ \begin{matrix}{y}_{t , 1 , 1} & & {y}_{t , M , 1} \\ ⋮ & … & ⋮ \\ {y}_{t , 1 , S} & & {y}_{t , M , S}\end{matrix} ] , \\ {Z}_{t} = [ \begin{matrix}{z}_{t , 1 , 1} & & {z}_{t , P , 1} \\ ⋮ & … & ⋮ \\ {z}_{t , 1 , S} & & {z}_{t , P , S}\end{matrix} ] \qquad$$

where N is the total number of variables in matrix Xₜ, M and P are the total number of variables for matrices Yₜ and Zₜ respectively. S is the number of participants and the matrices were measured at two time points t=1 and t=2.

Change measures across the two time points can be calculated as

ΔX=X₂−X₁

ΔY=Y₂−Y₁

ΔZ=Z₂−Z₁

### Two-Way Associations

Pair-wise associations were formed between all variables from the matrices ΔX, ΔY and ΔZ. Linear regression was performed between two variables and outliers were removed based on Cook's distance, a robust approach to remove outliers. After outlier removal, linear regressions were re-computed and all two-way associations with p≤0.05 were considered significant.

In what follows, Δyⱼ˜Δxᵢ will be used to denote the significant two-way association between any two variables.

### Three-Way Associations

Two-way associations with p≤0.05 were used to build three-way associations. In order to quantify three-way associations, the following steps (A-C, below) were performed

### Step A:

$$Δ {y}_{j} ∼ Δ {x}_{i} \{ \begin{matrix}∀ i ∈ \{ 1 , ⋯ , N \} \\ ∀ j ∈ \{ 1 , ⋯ , M \}\end{matrix}$$

Two-way associations were performed between variables Δyⱼ and Δxᵢ from matrices ΔY and ΔX.

### Step B:

$$Δ {z}_{k} ∼ Δ {y}_{j} \{ \begin{matrix}∀ j ∈ \{ 1 , ⋯ , M \} \\ ∀ k ∈ \{ 1 , ⋯ , P \}\end{matrix}$$

Two-way associations were performed between variables Δzₖ and Δyⱼ from matrices ΔZ and ΔY.

### Step C:

$$Δ {z}_{k} ∼ Δ {x}_{i} \{ \begin{matrix}∀ j ∈ \{ 1 , ⋯ , N \} \\ ∀ k ∈ \{ 1 , ⋯ , P \}\end{matrix}$$

Two-way associations were performed between variables Δzₖ and Δxᵢ from matrices ΔZ and ΔX.

Three-way associations between any three variables can be formed if the three steps above resulted in significant two-way associations for the common variables as below

Δxᵢ˜Δyⱼ˜Δzₖ˜Δxᵢ

### Permutation-Based Mediation Analysis

For mediation analysis linear regressions equations are defined between the independent variable (IV), dependent variable (DV) and mediator (M). Beta coefficients (β) and standard error (se) terms from the following linear regression equations are used to calculate the Sobel p-value and mediation effect percentage (T_{eff}) using the following steps:

M=β₀+β_{1A}(IV)+∈_{A} Step 1 (Path A):

DV=β₀+β_{1B}(M)+∈_{B} Step 2 (Path B):

DV=β₀+β_{1,1C}(IV)+∈_{1C} Step 3 (Path C, model 1):

DV=β₀+β_{1,2C}(IV)+β_{2,2C}(M)+∈_{2C} Step 4 (Path C, model 2):

Sobel's test can be used to test if β_{1,2C} was significantly lower than β_{1,1C} using the following equation:

$$Sobel z - score = \frac{( {β}_{1 , 1 C} - {β}_{1 , 2 C} )}{\sqrt{[ {( {β}_{2 , 2 C} )}^{2} {( {ϵ}_{1 A} )}^{2} ] + [ {( {β}_{1 A} )}^{2} {( {ϵ}_{2 C} )}^{2} ]}} \qquad ( 3 )$$

Using a standard 2-tail z-score table, the Sobel p-value is determined from Sobel z-score. Mediation effect percentage T_{eff} is calculated using the following equation:

$${T}_{e f f} = 100 * \frac{( {β}_{1 A} * {β}_{2 , 2 C} )}{( {β}_{1 A} * {β}_{2 , 2 C} ) + [ {β}_{1 , 1 C} - ( {β}_{1 A} * {β}_{2 , 2 C} ) ]} \qquad ( 4 )$$

Permutation-based mediation analysis was performed for the three-way associations following the steps listed below:
1. Mediation analysis was performed by assigning the original data variables Δxᵢ, Δyⱼ, Δzₖ as IV, DV and M to obtain reference Sobel z-score: z₀ and T_{eff}. Variables that formed three-way associations were considered.
2. Data permutation: values were randomly selected from x_{1,i} and x_{2,1} to assign to x_{1,i}′ and x_{2,i}′.
3. Across season measures were computed from the permuted dataset Δxᵢ′=x_{2,i}−x_{1,i}′. Similarly, Δyⱼ′ and Δzₖ′ were computed.
4. Mediation analysis was performed on the permuted dataset by assigning Δxᵢ′, Δyⱼ′, Δzₖ′ as IV, DV, and M; and the test statistic z_{q}′ was obtained.
5. The counter variable K was incremented by one if absolute value of z₀ was greater than absolute value of z_{q}′.
6. Steps 2-5 were repeated: q=1, 2, . . . , Q times.
7. Permutation-based p-value p_{Sobel}^{perm} was calculated as the proportion of the z_{q}′ values that are as extreme or more extreme than z₀—i.e., K/Q.
8. Mediation analysis was considered significant if p_{Sobel}^{perm}≤0.05 and T_{eff}>50%.

### Permutation-Based Moderation Analysis

For moderation analysis linear regression is defined between the independent variable (IV), dependent variable (DV) and moderator (M). The moderation is characterized by the interaction term between IV and M in the linear regression equation as given below:

DV=+β₀+β₁IV+β₂M+β₃(IV*M)+∈

Moderation can be significant if p_{β}_{₃}≤0.05 and p_{F}≤0.05, where p_{β}_{₃}≤0.05 indicates that β₃ is significantly different than zero using a t-test and p_{F} is the p-value associated with the overall F-test for the regression equation suggesting that the overall linear relationship is significant.

Permutation-based moderation analysis can include the following steps listed below:
1. Moderation analysis was performed by assigning the original data variables Δxᵢ, Δyⱼ, Δzₖ as IV, DV and M to obtain reference test-statistics: t₀ and F₀. Variables that formed three-way associations were considered.
2. Data permutation: values were randomly selected from x_{1,i} and x_{2,1} to assign to x_{1,i}′ and x_{2,i}′.
3. Across season measures were computed from the permuted dataset Δxᵢ′=x_{2,i}−x_{1,i}′. Similarly, Δyⱼ′ and Δzₖ′ were computed.
4. Moderation analysis was performed on the permuted dataset by assigning Δxᵢ′, Δyⱼ′, Δzₖ′ as IV, DV, and M; and the test statistics t_{q}′ and F_{q}′ were obtained.
5. The counter variable K₁ was incremented by one if absolute value of t₀ was greater than absolute value of t_{q}′.
6. K₂ was incremented by one if absolute value of F₀ was greater than absolute value of F_{q}′.
7. Steps 2-6 were repeated: q=1, 2, . . . , Q times. Here, Q=100,000.
8. Permutation-based p-value p_{β}_{₃}^{perm} was calculated as the proportion of the to values that are as extreme or more extreme than t₀—i.e. K₁/Q.
9. Permutation-based p-value p_{F}^{perm} was computed from F₀ and F_{q}′—K₂/Q.
10. Moderation analysis was considered significant if p_{β}_{₃}^{perm}≤0.05 and p_{F}^{perm}≤0.05.

Systems and methods in accordance with the present disclosure can use permutation-based mediation and moderation analysis for small sample sized studies that can integrate multi-scale datasets with several measures and provide control for the occurrence of false positives due to multiple hypothesis testing. These methods can draw inferences from the data directly rather than making assumptions about the underlying distribution of a small sample-sized dataset. In this way, the methods can maintain the irregularities of the observed dataset that are used to estimate the permutation probability. The permutation-based methods also has advantages over traditional multiple-testing correction methods like the Bonferroni correction, which can lead to unacceptable levels of false negatives resulting in exclusion of potentially relevant hypotheses. Using the permutation-based mediation and moderation analyses as described herein, associations between transcriptome, metabolome, brain imaging, and behavior measures were found for a dataset of contact sports athletes as a function of mechanical accelerations to the head. These methods helped identify unique metabolic biomarkers for subconcussive injury in contact sports athletes—these biomarkers may have been irrelevant using standard multiple comparison correction approaches for regression analyses. The results revealed from these analyses provided the first evidence in humans corroborating findings observed with animal research whose relevance was uncertain given the lack of human data; these human findings also matched closely known metabolomic and clinical abnormalities with genetic mutation illnesses in humans. The presented results demonstrated the usefulness of applying permutation procedures to mediation and moderation tests of multiple hypotheses and for controlling the rate of false positives. The techniques presented here provide a platform-independent tool relevant to a variety of datasets in diverse and interdisciplinary fields, such as biology and medicine, where integration of multi-scale data is utilized to unmask disease diagnosis and prognosis. As an example, this platform suggests an immediate path forward for research in mental health and some neurological illnesses where animal models are proving to not mirror the human conditions, and to not provide insight into human illnesses. There are emerging concerns that human centric research may be needed for dealing with mental illnesses such as depression and psychosis, and some neurological illnesses such as Parkinson's Disorder. This platform further allows an integration across omic measures or other molecular measures that can be readily manipulated for clinical intervention (which is less the case with genetics and epigenetics). Various methods described herein can be implemented using machine learning, such as to provide the variables described as inputs to a computational model that can be trained and executed according to the methods described herein.

### REFERENCE

1. Camargo A, Azuaje F, Wang H, Zheng H. Permutation—Based statistical tests for multiple hypotheses. Source Code Biol. Med. BioMed Central Ltd.; 2008. p. 15.
2. Belmonte M, Yurgelun-Todd D. Permutation testing made practical for functional magnetic resonance image analysis. IEEE Trans Med Imaging. 2001; 20:243-8.
3. Cook RD. Detection of Influential Observation in Linear Regression. Technometrics. Taylor & Francis Group; 1977; 19:15-8.

All or part of the processes described herein and their various modifications (hereinafter referred to as “the processes”) can be implemented, at least in part, via a computer program product, i.e., a computer program tangibly embodied in one or more tangible, physical hardware storage devices that are computer and/or machine-readable storage devices for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer (including a server) include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, e.g., magnetic, magneto-optical disks, or optical disks.

Computer program products are stored in a tangible form on non-transitory computer readable media and non-transitory physical hardware storage devices that are suitable for embodying computer program instructions and data. These include all forms of non-volatile storage, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks and volatile computer memory, e.g., RAM such as static and dynamic RAM, as well as erasable memory, e.g., flash memory and other non-transitory devices.

The construction and arrangement of the systems and methods as shown in the various embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced. Other substitutions, modifications, changes, and omissions may be made in the design, operating conditions and arrangement of embodiments without departing from the scope of the present disclosure.

As utilized herein, the terms “approximately,” “about,” “substantially”, and similar terms are intended to include any given ranges or numbers +/−10%. These terms include insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. A method for determining a biological and/or a psychological variable useful for illness or injury diagnosis, the method comprising:
receiving samples from a plurality of scales of organization in at least one of a body or a brain of one or more subjects;
generating a quantitative output using the samples;
determining that one or more compounds corresponding with the samples are increased or decreased relative to demographically matched normative controls responsive to the quantitative output;
determining that an individual has a variable profile similar to a subject having a target illness responsive to at least one of the determination of increase or decrease or the quantitative output;
integrating at least one first variable using a permutation-based mediation and moderation process to compute a first diagnostic likelihood;
integrating an output of performing the permutation-based mediation and moderation process with at least one second variable to at least one of update the first diagnostic likelihood or compute a second diagnostic likelihood; and
using a plurality of measures from multiple levels of spatio-temporal organization with machine learning to predict diagnosis.

2. A method of prognosing the longitudinal course of an illness or injury, the method comprising:
receiving samples from a plurality of scales of organization in at least one of a body or a brain of one or more subjects;
determining that one or more compounds corresponding with the samples are increased or decreased relative to demographically matched normative controls;
determining, responsive to the determination of increase or decrease, a longitudinal course of an individual when the person has a variable profile similar to that of a subject having an illness or injury for which a course of recovery is known;
integrating at least one first variable using a permutation-based mediation and moderation process to compute a prognostic likelihood;
integrating an output of performing the permutation-based mediation and moderation process with at least one second variable to compute a prognostic likelihood; and
using a plurality of measures from multiple levels of spatio-temporal organization with machine learning to predict prognosis for the longitudinal course of an individual with the illness or injury.

3. A method of assessing susceptibility for and resilience against an illness or injury, the method comprising:
receiving samples from a plurality of scales of organization in at least one of a body or a brain of one or more subjects;
generating a quantitative output using the samples;
determining that one or more compounds corresponding with the samples are increased or decreased relative to demographically matched normative controls;
determining, responsive to the determination of increase or decrease, that an individual has a variable profile in a range predicting susceptibility for and resilience against an illness or injury;
integrating at least one first variable using permutation-based mediation and moderation process to assess susceptibility for and resilience against an illness or injury;
integrating at least one second variable using an output of the permutation-based mediation and moderation process to assess susceptibility for and resilience against an illness or injury; and
using a plurality of measures from multiple levels of spatio-temporal organization with machine learning for assessing susceptibility for and resilience against an illness or injury.

4. A method of determining a treatment for an illness or injury, the method comprising:
receiving samples from a plurality of scales of organization in at least one of a body or a brain of one or more subjects;
generating a quantitative output using the samples;
determining that one or more compounds corresponding with the samples are increased or decreased relative to demographically matched normative controls;
determining, responsive to the determination of increase or decrease, that an individual has an illness or injury profile consistent with individuals for which a particular treatment of an illness or injury has satisfied a treatment criteria;
integrating at least one first variable using a permutation-based mediation and moderation process to assess optimal treatment for an illness or injury;
integrating at least one second variable with an output of the permutation-based mediation and moderation process to determine the optimal treatment for an illness or injury; and
using a plurality of measures from multiple levels of spatio-temporal organization with machine learning for determining the optimal treatment for an illness or injury.

5. A method of determining a target point in a pathway or process for identifying if a biopharmaceutical compound may minimize the metabolomic, transcriptomic, or proteomic abnormalities or other variables quantifying an illness or injury by:
quantifying if metabolomic measures are altered in a therapeutic, prognostic, predictive manner for individuals with an illness or injury;
determining if the biopharmaceutical compound alters metabolomic, proteomic, transcriptomic or other variables more than demographically matched normative controls;
assessing if the biopharmaceutical compound affects a plurality of measures so an individual has a metabolomic, proteomic, transcriptomic or other variables profile consistent with individuals that have responded well to a particular treatment of that illness or injury;
testing the biopharmaceutical compound against integrated variable indices for optimal treatment of an illness or injury;
testing the biopharmaceutical compound against metabolomic, proteomic, transcriptomic or other variable data with hormone measures (e.g., progesterone) for optimal treatment of an illness or injury;
testing the biopharmaceutical compound against metabolomic, proteomic, transcriptomic or other variable data with genotype data (e.g., a SNP at DARC or TPH2 or KIAA0319) for the optimal treatment of an illness or injury; and
testing the biopharmaceutical compound against a plurality of measures from metabolomic, transcriptomic, proteomic, hormone, genetics data with machine learning for optimal treatment of an illness or injury.

6. A system, comprising:
one or more processors configured to perform one or more steps of claim 1.

7. A system, comprising:
one or more processors configured to perform one or more steps of claim 2.

8. A system, comprising:
one or more processors configured to perform one or more steps of claim 3.

9. A system, comprising:
one or more processors configured to perform one or more steps of claim 4.

10. A system, comprising:
one or more processors configured to perform one or more steps of claim 5.
